(19) United States
(12) Patent Application Publication
(10) Pub. No.: US 2020/0060972 A1
(43) Pub. Date: Feb. 27, 2020

(54) FORMULATIONS OF POLYINOSINIC ACID AND POLYCYTIDYLIC ACID FOR THE PREVENTION OF UPPER RESPIRATORY TRACT INFECTIONS

(71) Applicant: Janssen Sciences Ireland Unlimited Company, Co Cork (IE)

(72) Inventors: Bruce Malcolm, British Columbia (CA); Rainer Wechselberger, Drebber (DE)

(21) Appl. No.: 16/461,631

(22) PCT Filed: Nov. 15, 2017
(86) PCT No.: PCT/IB2017/001516

§ 371 Date: May 16, 2019

Related U.S. Application Data
(60) Provisional application No. 62/422,873, filed on Nov. 16, 2016.

(51) Int. Cl. A61K 9/16; A61K 31/713; A61K 9/00; A61P 31/16

(52) U.S. Cl. CPC A61K 9/1652; A61K 31/713; A61K 9/1617; A61K 9/1694; A61K 9/007; A61P 31/16; A61K 9/0043

(57) ABSTRACT

Provided herein are microparticles and compositions comprising Poly(I:C) and/or polyinosinic acid (Poly I) and polycytidylic acid (Poly C) for use in preventing viral infections of the upper respiratory tract, such as human rhinovirus infection or an influenza virus infection. A nasal delivery device comprising a composition of the invention is also described.

### CROSS-REFERENCE TO RELATED APPLICATION

This application claims the benefit of priority to U.S. Provisional Patent Application Ser. No. 62/422,873, filed Nov. 16, 2016, which is hereby incorporated by reference herein in its entirety.

### BACKGROUND

The common cold (also known as nasopharyngitis, acute viral rhinopharyngitis, acute coryza, or a cold) is an infectious disease of the upper respiratory system caused primarily by viruses, similar to influenza or influenza-like illnesses.

In total, over 200 known serologically different viral types cause colds. The most commonly implicated virus is the rhinovirus (30-50%), a type of picornavirus with 99 known serotypes. Others include coronavirus (10-15%), influenza (5-15%), human parainfluenza viruses, human respiratory syncytial virus, adenoviruses, enteroviruses, and metapneumovirus.

For example, coronaviruses are known to result in adult colds. However, while over 30 coronaviruses have been identified, only 3 or 4 are known to cause infections in humans. In addition, these viruses are difficult to culture in vitro to gain insight into their function. Due to the many different types of viruses and their tendency for continuous mutation, robustly effective prevention of many viral diseases has been a largely intractable challenge. In addition, research has yet to develop a standard vaccine prophylaxis that would be effective against all of them.

Viral replication of cold viruses in humans typically begins 2 to 6 hours after initial contact. In some cases, the patient is infectious for a couple of days before the onset of symptoms. Symptoms usually begin about 2 to 5 days after initial infection. The common cold is most infectious during the first two to three days of symptoms. There is currently no known treatment that shortens the duration of a cold; however, symptoms usually resolve spontaneously in about 7 to 10 days, with some symptoms possibly lasting for up to three weeks. The virus may still be somewhat infectious until symptoms have completely resolved.

Another pervasive viral infection is caused by human rhinovirus (HRV), which is a member of the Enterovirus genus in the Picornaviridae family. HRVs can infect the upper and lower airways, nasal mucosa, sinuses and middle ear, and infections produce symptoms of “the common cold”. Infections are self-limiting and are typically restricted to the upper airways.

There are no commercial antiviral agents for the treatment or prevention of rhinovirus infections or common colds. Treatment of upper respiratory tract infections are based upon management of the symptoms (sneezing, nasal congestion, rhinorrhea, eye irritation, sore throat, cough, headaches, fever, chills) typically using over the counter sleep-inducing oral antihistamines, aspirin, cough suppressants, and nasal decongestants. Symptomatic treatment generally involves using anti-histamines and /or vaso-constrictive decongestants that have stimulant side-effects.

Airway epithelial cells are the primary target of upper respiratory tract (URT) infective agents like rhino- and corona viruses. As infection with these viruses occurs prior to the onset of symptoms that indicates immune system response, direct antiviral therapeutic intervention is unlikely to prove very effective. However, treatment with an antiviral agent that stimulates an immune response that mimics the response to viruses could enable the patient's immune system to activate and effectively prevent infection. A patient would not develop symptoms and also would not be a carrier of the virus. Some viral infections are asymptomatic in one person but infectious in another. In these cases, transmission of the virus can be widespread as the infected person does not appear ill. Transmission is particularly detrimental in schools, hospitals, nursing homes and others with susceptible populations living in close quarters. The human and monetary costs of viral infection symptoms and treatment can be prevented with an effective prophylactic anti-viral agent that is administered prior to infection.

Thus, there is a need for a broadly effective, convenient, side-effect free prophylactic that would alleviate viral infections of the upper respiratory system.

### SUMMARY

Provided herein is a microparticle comprising polyinosinic acid and polycytidylic acid mixed with one or more carrier polymers. In certain embodiments, the polyinosinic acid and polycytidylic acid are each about 300 to about 6,000 bases long. In certain embodiments, the one or more carrier polymers comprise one or more of starch, hyaluronate, alginate, carboxymethyl cellulose, microcrystalline cellulose, or dipalmitoylphosphatidylcholine.

Provided herein is a composition comprising a plurality of microparticles as described herein. Disclosed herein are methods for preventing upper respiratory infections, comprising administering to a subject a microparticle or composition as provided herein. In certain embodiments, the viral infection can be a human rhinovirus infection or an influenza virus infection. The compositions can be nasally administered to a subject, such as by using a nasal delivery device.

### BRIEF DESCRIPTION OF THE DRAWINGS

FIG. 1 depicts the IFN-β signal induction in mice who were nasally administered a composition of Poly I, Poly C, Poly(I:C) or mixed Poly I+Poly C using a spatula.

FIG. 2 depicts the IFN-β signal induction in mice who were nasally administered a composition of Poly I, Poly C, Poly(I:C) or mixed Poly I+Poly C using a tip device.

FIG. 3A and FIG. 3B depict the IFN-β signal induction in male mice (FIG. 3A) and female mice (FIG. 3B) who were nasally administered a composition of Poly I, Poly C, Poly(I:C) or mixed Poly I+Poly C initially in the left nostril only, with male mice in FIG. 3A receiving an additional dose on their nose-tip.

FIG. 4A depicts the sum of the IFN-β signal induction data shown in FIGS. 1, 2, 3A and 3B in a logio scale. FIG. 4B depicts the sum of the IFN-β signal induction data shown in FIGS. 1, 2, 3A and 3B on a linear scale.

FIG. 5 depicts the dose response data of administration of standard Poly(I:C) in female and male mice after 6 h.

FIG. 6A depicts the kinetics of IFN-β induction after administration of standard Poly(I:C), and FIG. 6B depicts the kinetics of IFN-β induction after administration of LMW Poly(I:C). FIG. 7A depicts the dose response curve for standard Poly(I:C) and LMW Poly(I:C) 3 hours after dosing. FIG. 7B depicts the dose response curve for standard poly(I:C) and LMW Poly(I:C) 6 hours after dosing. FIG. 7C depicts the dose response curve for standard Poly(I:C) and LMW Poly(I:C) 24 hours after dosing.

FIG. 8A depicts the IFN-β reporter induction at the nose in wt IFN-β reporter mice as well as in reporter mice lacking IRF3, IRF7 or IFN-β. These mice were dosed with 30 standard Poly(I:C). FIG. 8B depicts the IFN-β reporter induction at the nose in wt IFN-β reporter mice as well as in reporter mice lacking IRF3, IRF7 or IFN-β. These mice were dosed with 300 μg LMW Poly(I:C).

FIG. 9A and FIG. 9B depicts the radiance of a region of interest (ROI) for mice administered microparticles comprising Poly(I:C) and pea starch (Lycoat RS780®) at a ratio of 1:3 (Poly(I:C)/Lycoat RS780 1/3), 1:5 (Poly(I:C)/Lycoat RS780 1/5), or 1:12 Poly(I:C)/Lycoat RS780 1/12), as well as a negative control (Placebo starch Lycoat RS780). The mice comprised a firefly luciferase gene in the interferon beta (INF-β) locus, and thus, radiance correlated with INF-β expression. FIG. 9 A depicts the data plotted with a linear y-axis and FIG. 9B with a logarithmic y-axis.

FIG. 10A depicts images of mice comprising a gene for firefly luciferase in the interferon beta (INF-β) locus that were administered luciferin 2-3 hours prior to administering microparticles comprising Poly(I:C) and pea starch (Lycoat RS780®). FIG. 10B depicts images of the mice 24 hours after administering microparticles.

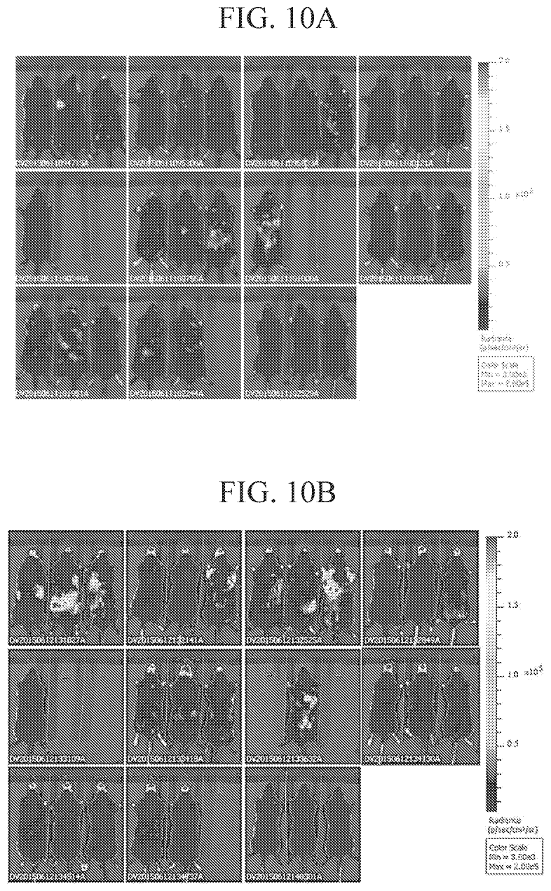

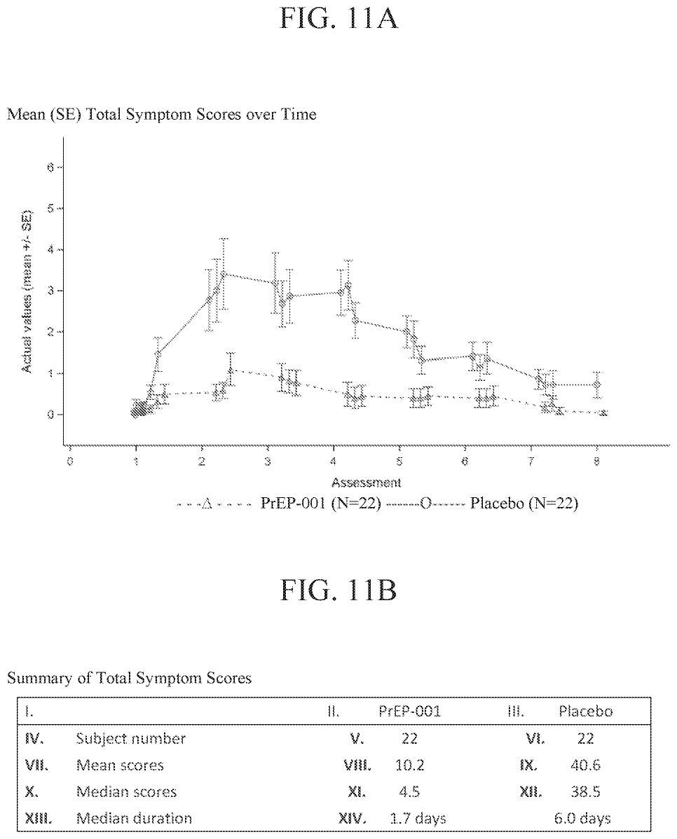

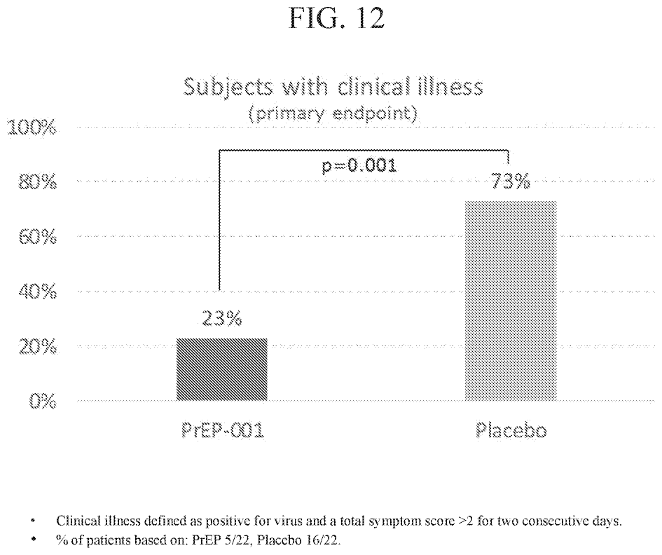

The top row of four images in both FIG. 10A and FIG. 10B are replicates of mice that were administered microparticles comprising Poly(I:C) and pea starch (Lycoat RS780®) at a ratio of 1:3 (left mouse in each image), 1:5 (center mouse in each image), or 1:12 (right mouse in each image). The middle row of four images depicts two control mice, which were each imaged separately (left image and right-center image), and two images of three mice each (left-center image and right image), which were ordered as in the top row. The bottom row of three images consists of mice that were administered microparticles comprising Poly(I:C) and pea starch (Lycoat RS780®) at a ratio of 1:3 (left image and center image, each depicting three mice) and mice that were administered microparticles comprising solely pea starch (Lycoat RS780®; right image, depicting three mice).

FIG. 11A depicts the mean total symptom scores for subjects who received PrEP-001 vs. placebo. FIG. 11B depicts a summary of the scores and duration subjects experienced symptoms.

FIG. 12 depicts the percentage of subjects having clinical illness at the primary endpoint comparing subjects who received PrEP-001 vs. placebo.

### DETAILED DESCRIPTION

### Immune System Activation

Toll-like receptor 3 (TLR3) is a protein that in humans is encoded by the TLR3 gene. TLR3 is a member of the Toll-like receptor family of pattern recognition receptors of the innate immune system, which plays a fundamental role in pathogen recognition and activation of innate immunity. TLRs are highly conserved from Drosophila to humans and share structural and functional similarities. They recognize pathogen-associated molecular patterns (PAMPs) that are expressed on infectious agents, and mediate the production of cytokines necessary for the development of effective immunity. The various TLRs exhibit different patterns of expression. The TLR3 receptor is also expressed by airway epithelial cells and is restricted to the dendritic subpopulation of the leukocytes.

TLR3 recognizes double-stranded RNA (dsRNA), such as that present in viruses. Double-stranded RNA is RNA with two complementary strands that can be formed during the viral replication cycle. Upon recognition, TLR3 induces the activation of transcription factors like NF-κB and Interferon Regulatory Factor 3 (IRF3) to increase production of type I interferons which signal other cells to increase their antiviral defenses.

The tertiary structure of TLR3 forms a large horseshoe shape that contacts another nearby TLR3, forming a “dimer” of two horseshoes. Much of the TLR3 protein surface is covered with sugar molecules, making it a glycoprotein, but on one face (including the proposed interface between the two horseshoes), there is a large sugar-free surface. This surface also contains two distinct patches rich in positively-charged amino acids, which may be a binding site for negatively-charged double-stranded RNA.

A double-stranded RNA polymer of inosinic acid complexed to a polymer of cytidylic acid (Poly(I:C) has demonstrated prophylactic efficacy. Polyinosinic-polycytidylic acid (Poly(I:C)) is a dsRNA with a MW distribution up to, for instance 3,600,000 Daltons. Poly(I:C) is a Toll Like Receptor 3 (TLR3) ligand that mimics viral RNA and is a known stimulant of the innate immune response. When administered to the nasal mucosa, it induces expression of anti-viral proteins like interferon α and β (IFNs) in the nasal epithelium. The IFNs then proceed to induce the expression of Interferon Stimulated Genes (ISGs) that further contribute to the antiviral state in the cell and stimulate adjacent cells to activate their innate response as well. Poly(I:C) has a higher efficiency in activating this pathway than purified dsRNA from viral sources. Poly(I:C) is further known as activator of the retinoic acid inducible gene 1 (RIG-I) receptor and the melanoma differentiation-associated gene-5 (MDA5) (a RIG-I like receptor) located in the cytosol, both involved in a similar pathway of innate immune response.

### Microparticles and Their Compositions

To improve patient compliance and reduce the frequency of dosing, provided herein are microparticles that comprise single-stranded polyinosinic acid (Poly I) and single-stranded polycytidylic acid (Poly C) that are not associated by hydrogen bonding or covalent bonding at the time of administration. Upon administration to a moist mucosal surface, uncomplexed Poly I and Poly C can form complexed Poly(I:C) and thus prime the innate immune system and provide protection against viral infection. These microparticles and their compositions are convenient and effective in creating formulations for administration.

Provided herein is a microparticle comprising polyinosinic acid and polycytidylic acid mixed with one or more carrier polymers. In certain embodiments, the polyinosinic acid and polycytidylic acid are each about 300 to about 6,000 bases long.

In certain embodiments, the one or more carrier polymers comprise one or more of starch, hyaluronate, alginate, carboxymethyl cellulose, microcrystalline cellulose, or dipalmitoylphosphatidylcholine. In certain embodiments, the polyinosinic acid and polycytidylic acid are each about 300 to about 6,000 bases long.

Provided herein is a microparticle comprising polyinosinic acid, polycytidylic acid, and one or more carrier polymers comprising a pregelatinized starch or a partially pregelatinized starch. In certain embodiments, the pregelatinized starch or a partially pregelatinized starch is partially pregelatinized maize starch, pregelatinized pea starch, or pregelatinized potato starch.

In certain embodiments, the pea starch is pregelatinized hydroxypropyl pea starch. In some embodiments, the microparticle may include water. In some embodiments, the amount of water in the microparticle ranges from about 3% to about 8%. In certain embodiments, a microparticle may consist of polyinosinic acid, polycytidylic acid, starch, and water. In some embodiments, the microparticle consists essentially of polyinosinic acid, polycytidylic acid, one or more carrier polymers, and water.

Also provided herein is a microparticle consisting of polyinosinic acid mixed with one or more carrier polymers and water. In certain such embodiments, the carrier polymer is not chitosan. Certain embodiments provide a microparticle consisting of polycytidylic acid mixed with one or more carrier polymers and water. In certain such embodiments, the carrier polymer is not chitosan. In addition, provided herein is a microparticle, comprising polyinosinic acid and one or more carrier polymers comprising pea starch, pregelatinized potato starch, microcrystalline cellulose, and hyaluronate. Certain embodiments provide a microparticle comprising polycytidylic acid, and one or more carrier polymers comprising pea starch, pregelatinized potato starch, microcrystalline cellulose, and hyaluronate. In certain embodiments, provided herein is an equimolar mixture of microparticles consisting of polyinosinic acid and microparticles consisting of polycytidylic acid.

Poly I and Poly C are single stranded non-natural RNA polymers that are each typically present as their sodium salt under physiologic conditions. The molecular formula of Poly I is (C₁₀H₁₀N₄NaO—P)ₓ and of Poly C is (C₉H₁₁NaN₃O—P)ₓ. In certain preferred embodiments, the average chain length for each of the Poly I and Poly C strands ranges between 300 to 6,000 bases, corresponding to approximately 99 kDa to 1,981 kDa for Poly I and 92 kDa to 1,831 kDa for Poly C. In even more preferred embodiments, the average chain length for each of the Poly I and Poly C strands ranges between 500 to 2,000 bases, corresponding to approximately 16.5 kDa to 660 kDa for Poly I and 15.3 kDa to 610 kDa for Poly C.

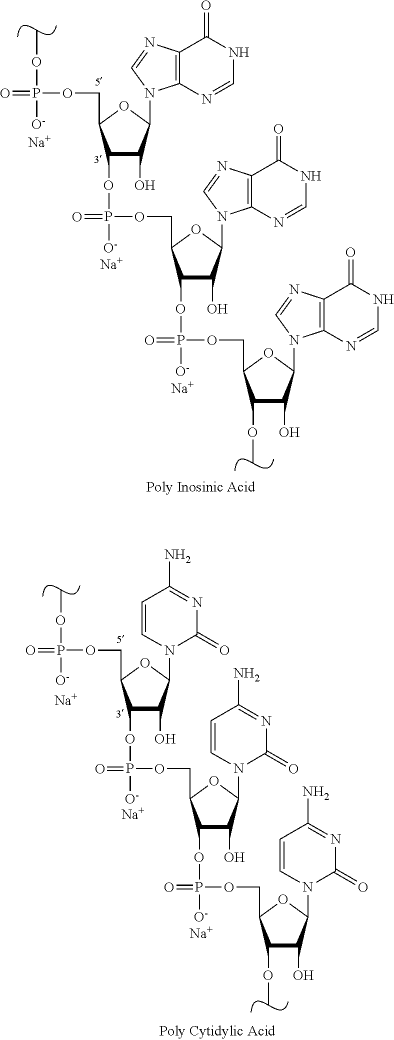

Poly I and Poly C can be synthesized by individually polymerizing the nucleoside diphosphates inosine and cytidine in the presence of polynucleotide phosphorylase (PNPase). Each nucleoside diphosphate is individually polymerized, e.g., by PNPase for 20-24 hrs. to control the length of the resulting ribonucleic acid polymer, to provide homopolymeric strands. The enzyme, protein kinase, can be used to terminate the polymerization reaction. The resulting homopolymers (i.e., single-stranded RNA molecules) may then hydrolyzed to control the molecular weight range of each polymer product within a specified range. The hydrolyzed product can be treated with ethanol to precipitate the single stranded RNA molecules (ssRNA) from solution. The precipitate may be separated from the supernatant and dissolved in water. The solution of ssRNA may then be filtered to remove particulates, ultra filtered to remove the low-molecular weight contaminants and then lyophilized. Lyophilized ssRNA products can be individually tested for purity, molecular weight, and other quality attributes to ensure the products are within specification. The single stranded Poly I and single stranded Poly C when they occur together are referred to herein as “Poly(I+C)”.

Compositions and formulations according to the present invention can be prepared in a wide variety of ways. In certain embodiments, the microparticles are formed using particle formation process, such as the spray dry process described in WO2013/164380, incorporated herein by reference in its entirety. In some embodiments, Poly(I+C) is spray dried from an aqueous mixture containing the carrier polymer, Poly I and Poly C. In other embodiments, Poly (I/C) is spray dried from an aqueous mixture containing the carrier polymer, Poly I and Poly C.

In certain embodiments, the RNA component of the microparticles is single stranded Poly I and single stranded Poly C. This composition can be formed by separately adding Poly I and Poly C to the carrier polymer, or by adding Poly(I:C) to the carrier polymer under conditions that disfavor complexation of Poly(I:C) and induce dissociation into the individual strands. When these compositions are administered nasally, the ionic strength of the aqueous coating of the nose mucosa promotes the annealing of the single-stranded Poly I and Poly C to form double-stranded RNA (known as Poly(I:C)), which is capable of activating biological pathways that stimulate an immune response. Pulmonary or respiratory administration can also promote annealing to form Poly(I:C) upon contact with the aqueous mucosa of the lung.

In some embodiments, the Poly I and Poly C may each be present individually in microparticles of the composition (i.e., some particles comprise Poly I but not Poly C, others Poly C but not Poly I). Provided herein is a composition comprising a plurality of microparticles comprising polyinosinic acid and one or more carrier polymers, and a plurality of microparticles comprising polycytidylic acid and one or more carrier polymers. For such compositions, upon administration to a mucosal surface, dissolution of the particles allows Poly I and Poly C to come into contact with each other and complex to form the active Poly(I:C). In such compositions, smaller particle sizes are preferred to favor the formation of Poly(I:C) on dissolution of the individual particles.

The compositions and microparticles disclosed herein typically do not have appreciable amounts of Poly(I:C) in the microparticle, but enable its formation upon delivery to the nasal mucosa. Poly(I:C) can dissociate and/or form in a composition depending on the other components of the composition and properties such as ionic strength. A Poly (I/C) mixture can also form from dissociation of Poly (I:C) or from complexation of Poly I and Poly C. Accordingly, references to a composition that comprises Poly(I:C) should be understood as a convenience, and may include a composition in which the Poly I and Poly C are present in a dissociated state but can recomplex in a suitable environment, such as a mucosal surface (e.g., of the nose or lung).

Accordingly, in an exemplary method for preparing microparticles as disclosed herein, an aqueous solution may be prepared by combining one or more carrier polymer(s), Poly I, and Poly C (in appropriate ratios as discussed elsewhere herein with respect to the formulations) with water, such as demineralized water. The resulting solution can then be fed into a spray dryer to form microparticles as described herein. Similarly, an aqueous solution may be prepared by combining one or more carrier polymer(s) and Poly(I:C) (in appropriate ratios as discussed elsewhere herein with respect to the formulations) with water, such as demineralized water. Likewise, this solution can be fed into a spray dryer to form microparticles as described herein.

In yet another method, each of Poly I and Poly C can be separately combined with one or more carrier polymers and water, such as demineralized water, to form two aqueous solutions, one comprising Poly I and the other comprising Poly C. These solutions can be spray-dried individually, or can be combined and sprayed through a single nozzle. If spray-dried individually, the resulting particles can be mixed to provide a composition comprising particles of both Poly I and Poly C, which can, upon contact with the moist environment of nasal or lung mucosa, combine to form Poly(I:C).

In some embodiments, the ratio of Poly(I+C) to Poly(I:C) in Poly(I/C) can be about 1000:1, about 500:1, about 100:1, or about 10:1.

In certain embodiments, the compositions are administered nasally. In certain embodiments, the carrier polymers are water soluble and have a low viscosity to promote even consistency in the compositions, and may also promote adherence of the disclosed formulations to a mucosal surface without interfering with a) the formation of complexed Poly(I:C) or b) the uptake of the complexed Poly(I:C) by a mucosal surface. The compositions also have increased stability as a dry powder and in liquid formulations, which enables dosing regimens that can be easily adhered to by patients.

In some embodiments, the carrier polymers can be cationic, neutral or anionic. In certain embodiments, upon contact with the nasal mucosa during administration, the carrier polymer serves to create an aqueous environment with sufficient ionic strength so the Poly I and Poly C anneal to form the active Poly(I:C) dsRNA that fosters the immunogenic response. In other embodiments, the microparticle delivers Poly I and Poly C upon administration and the aqueous environment of the nasal mucosa has sufficient ionic strength to induce annealing to give Poly(I:C) dsRNA. If the aqueous mucosal environment lacks sufficient ionic strength, the Poly I and Poly C ssRNA polymers will remain single-stranded, even if present in the same microparticle.

The ionic strength of a solution is a measure of the concentration of ions or electrolytes in that solution. Ionic compounds, when dissolved in water, dissociate into ions. The total electrolyte concentration in solution can affect the dissociation or the solubility of different salts. One of the main characteristics of a solution with dissolved ions is the ionic strength. Ions can come from inorganic or organic salts of acids and bases, and also from charged polymers of biological or synthetic origin. In some embodiments, the ion source is a molecule that is zwitterionic. In physiology, common electrolytes include, but are not limited to, sodium (Na⁺), potassium (K⁺), calcium (Ca²⁺), magnesium (Mg²⁺), chloride (Cl⁻), fluoride (F⁻), phosphate (PO₄³⁻) hydrogen phosphate (HPO₄²⁻), and hydrogen carbonate (HCO₃⁻) which form from salt dissociation in aqueous media. Other biological ions include, but are not limited to, acetate (CH₃CO₂⁻), sulfate (SO₄²⁻), hydroxide (OH⁻), ammonium (NH₄⁻), iron (Fe²⁺ and Fe³⁺), quaternary ammonium (N_{R}⁴⁺ with R being alkyl or aryl), carbonate (CO₃²⁻), hydrogen carbonate (HCO₃⁻), citrate (HOC(COO⁻)(CH₂COO⁻)₂, cyanide (CN⁻), nitrate (NO³⁻), and nitrite (NO²⁻).

In certain embodiments, the carrier polymer provides a beneficial consistency for creating the microparticles using a spray dry process. For example, to prepare a dry powder composition for, e.g., intranasal administration, drum dried waxy maize starch has a dual function: (1) to act as a bio-adhesive in the nose, and (2) the amylopectin present in high concentration in waxy maize starch is broken down by amylases in the nose to release the Poly I and the Poly C. Starches with high amylopectin content or with chemically modified starches exhibit good muco-adhesion to nasal tissues. Other exemplary carrier polymers include Na-Alginate, partially pregelatinized maize starch, DPPC and carboxymethyl cellulose. Compositions having these carrier polymers showed interferon stimulating activity in an interferon-promoter-GFP-reporter (A549-IFN-GAR5, clone H10) cell line assay. However, unexpectedly, compositions having carbopol, κ-carrageenan, chitosan, or polyethylamine the carrier polymer inhibited or completely blocked the interferon stimulating capacity. See, e.g., WO2013/164380.

As used herein, the term “starch” refers to a polymeric carbohydrate having individual glucose units joined by glycosidic bonds. The number of glucose units can range from about 300 to about 1,000. Starch has two types of molecules: the linear and helical amylose and the branched amylopectin. Depending on the plant, starch generally contains about 20% to about 25% amylose and about 75% to about 80% amylopectin by weight. Although in absolute mass only about one quarter of the starch in plants consist of amylose, there are about 150 times more amylose molecules than amylopectin molecules. Amylose is a smaller molecule than amylopectin.

Starch molecules arrange themselves in plants in semi-crystalline granules. Each plant species has a unique starch granular size: rice starch is relatively small (about 2 μm) while potato starches have larger granules (up to about 100 μm). Starch becomes soluble in water when heated. The granules swell and burst, the semi-crystalline structure is lost and the smaller amylose molecules start leaching out of the granule, forming a network that holds water and increasing the mixture's viscosity. This process is called starch gelatinization. Untreated starch requires heat to thicken or gelatinize. When a starch is pre-cooked, it can thicken instantly in cold water. This is referred to as a pregelatinized starch.

In some preferred embodiments, the starch is a pregelatinized starch. For example, in certain, preferred embodiments, the starch may be pregelatinized pea starch or pregelatinized maize starch (e.g., pregelatinized waxy maize starch). Starch gelatinization may increase the solubility of the starch.

Main sources of starches have their plant origins in rice, wheat, maize/corn, potatoes, and cassava (i.e., tapioca starch). Non-limiting examples of additional starch sources include acorns, arrowroot, arracacha, bananas, barley, breadfruit, buckwheat, canna, colacasia, katakuri, kudzu, malanga, millet, oats, oca, polynesian arrowroot, sago, sorghum, sweet potatoes, rice, rye, taro, chestnuts, water chestnuts and yams, and many kinds of beans, such as favas, lentils, mung beans, peas, and chickpeas.

Starches can also be chemically modified to alter their physical properties, for example, to withstand conditions frequently encountered during processing or storage, such as high heat, high shear, low pH, freeze/thaw and cooling. Modification may also alter the hydrophobicity, hydrophilicity, charge, hygroscopicity, viscosity, and/or solubility of the starch. Modified starches include, but are not limited to, dextrin, acid-treated starch, alkaline-treated starch, bleached starch, oxidized starch, enzyme-treated starch, monostarch phosphate, distarch phosphate, phosphated distarch phosphate, acetylated distarch phosphate, starch acetate, acetylated distarch adipate, hydroxypropyl starch, hydroxypropyl distarch phosphate, hydroxypropyl distarch glycerol, starch sodium octenyl succinate, starch aluminum octenyl succinate, acetylated oxidized starch, cationic starch, hydroxyethyl starch, and carboxymethylated starch.

In certain embodiments, the carrier polymer comprises one or more of starch, alginate, carboxymethyl cellulose or DPPC (dipalmitoylphosphatidylcholine). The starch may be sourced from, e.g., maize, potato or cassava. In some embodiments, the starch is partially pregelatinized maize starch. In some embodiments, the carrier polymer is alginate, and the alginate is sodium alginate. In other embodiments, the carrier polymer is dipalmitoylphosphatidylcholine.

In certain embodiments, the carrier polymer comprises pea starch, pregelatinized potato starch, microcrystalline cellulose, or hyaluronate. In certain embodiments, the pea starch is pregelatinized hydroxypropyl pea starch. In other embodiments, pregelatinized hydroxyethyl pea starch can be used. For example, pea starch shows a surprisingly less sticky behavior to the inside of a vial, tube or device (like sprays) when such vials, tubes or devices are filled with the disclosed compositions compared to other starches that may be used for the same purpose. See, e.g., WO2015/067632. These compositions can be more precisely dosed and administered to the patient in need, since less composition will stick to the inner side of the nasal spray device accordingly. Starches with high amylopectin content or with chemically modified starches exhibit good muco-adhesion. In addition, the formulation enhances the efficacy of Poly I and Poly C and permits much less frequent dosing with even greater TLR3 stimulating activity.

In certain embodiments, the carrier polymer can be a starch selected from maize starch (i.e., cornstarch), wheat starch, potato starch, and pea starch. The starch may be selected from banana starch, rice starch, barley starch, rye starch, millet starch, oat starch, yam starch, sweet potato starch, cassava starch (i.e., tapioca starch), sago starch, arrowroot starch, fava bean starch, lentil starch, mung bean starch, and chickpea starch. The starch may comprise starch from more than one source, e.g., the starch may comprise pea starch, potato starch, wheat starch, and/or maize starch. Each of the above starches and combinations thereof are interchangeable with pea starch in each embodiment of the invention, and pea starch may be substituted with starch of any origin (or combinations of starches) in any embodiment of the invention. In preferred embodiments, the starch comprises a pea starch, such as pregelatinized hydroxypropyl pea starch, or even consists or consists essentially of a pea starch, such as pregelatinized hydroxypropyl pea starch (e.g., Lycoat RS780®). In some preferred embodiments, the starch comprises a maize starch (e.g., waxy maize starch), or even consists essentially of a maize starch (e.g., waxy maize starch).

Some carrier polymers can be used in derivative form, such as hyaluronate as its sodium salt. In certain embodiments, the pea starch is derivatized as hydroxypropylated pregelatinized pea starch (chemically modified), as this material is cold water-swelling and contains a cold water-soluble fraction, resulting in a homogeneous dispersion when mixed at low shear with Poly(I:C) and/or Poly I and Poly C. The resulting starch dispersions have a low to medium viscosity which allows spray drying into a homogeneous powder. Some carrier polymers also show beneficial properties based on the source material, such as using pea starch isolated from the pea plant genus Lathyrus.

In some embodiments, the starch may be a modified starch as described herein. In some embodiments, 0% to about 40% of the hydroxyl groups of the starch are hydroxypropylated, such as about 1% to about 20%, or about 2% to about 10%. In some embodiments, 0% to about 40% of the hydroxyl groups of the starch are hydroxyethylated, such as about 1% to about 20%, or about 2% to about 10%. In certain preferred embodiments, the starch is a hydroxypropyl starch, such as a hydroxypropyl pea starch. In certain preferred embodiments, the starch is a hydroxyethyl starch, such as hydroxyethyl maize starch.

The microparticles disclosed herein comprise one or more carrier polymers. In certain embodiments, the microparticle has a single carrier polymer. In other embodiments, the microparticle has two or more carrier polymers. For example, a microparticle may comprise one starch and one alginate, thus having one polymer from each type. In some embodiments, a microparticle may comprise two polymers of the same type, such as pea starch and potato starch. Other non-limiting embodiments include a microparticle comprising one or more modified starches, such as dextrin and pregelatinized hydroxypropyl pea starch. This disclosure contemplates microparticles having any disclosed carrier polymer as a single polymer in the microparticle, and microparticles having any two or more disclosed carrier polymers in the microparticle.

Provided herein are compositions comprising microparticles as described herein. In certain embodiments, the compositions can be in the form of a dry powder. In other embodiments, the compositions can be in the form of a bi-phasic suspension, where the wherein the organic solvent is based on glycerol or ethanol or a combination thereof. In certain embodiments, the composition is an aqueous solution, such as a solution in water or in phosphate-buffered saline (PBS). In other embodiments, the composition is a liquid having an organic solvent selected from one or more of glycerol, ethanol, trifluoranes, or other etherous propellants.

Commercial sources of “Poly(I:C)” are often a lyophilized powder of a mixture of single-stranded Poly I and single-stranded Poly C. When reconstituted in water, Poly I and Poly C remain single-stranded. However, if the powder is dissolved in a liquid high in sodium content (>100 mM NaCl) such as about 3% to about 6% PBS, the increased ionic strength induces the two strands to anneal and form Poly(I:C) in solution.

### Microparticle Composition Ratios

In some embodiments, a single carrier polymer is used in forming the microparticle. In other embodiments, multiple carrier polymers are used and added separately to the Poly(I/C) mixture prior to forming the microparticle. In other embodiments, the multiple carrier polymers are admixed together prior to addition to the Poly(I/C) mixture prior to forming the microparticle. The ratios of Poly I and Poly C to carrier polymer disclosed herein and below apply equally to the ratio of Poly(I/C) to the sum of all carrier polymers in the microparticle. For example, if both DPPC and pea starch carrier polymers are present, a ratio of “Poly(I/C) to carrier polymer of about 1/200 (w/w)” equally discloses a 1 part Poly(I/C) to 200 parts of the sum of the DPPC and pea starch.

In certain embodiments, the ratio of the combination of Poly(I/C) to carrier polymer can range from about 1/200 (w/w) to 1/0.1 (w/w), such as from about 1/100 (w/w) to 1/5 (w/w), further such as from about 1/12 (w/w) and 1/9 (w/w). In certain embodiments where the microparticle includes pea starch, the ratio of the Poly(I/C) to pea starch can be about 1:3.

In preferred embodiments, the microparticle has a ratio of a combination of Poly(I/C) to carrier polymer of about 1:1, about 3:1, about 10:1, about 30:1, about 50:1, about 75:1, or about 100:1. In some embodiments, the microparticle has a ratio of a combination of Poly(I/C) to carrier polymer ranging from about 3:1 to about1:1. In other embodiments, the microparticle has a ratio of a combination of Poly(I/C) to carrier polymer of about 0.1:1, about 0.3:1, about 0.5:1, about 0.75:1 or about 1:1. In some embodiments, the microparticle has a ratio of a combination of Poly(I/C) to carrier polymer of about 1:1.

In preferred embodiments, the microparticle has a ratio of a combination of Poly(I/C) to carrier polymer of greater than about 1:9. For example, the microparticle has a ratio of a combination of Poly(I/C) to carrier polymer of about 1:1 to about 1:8, about 1:1 to about 1:7, about 1:1 to about 1:6, about 1:1 to about 1:5, about 1:1 to about 1:4, about 1:1 to about 1:3, about 1:2 to about 1:8, about 1:2 to about 1:7, about 1:2 to about 1:6, about 1:2 to about 1:5, about 1:2 to about 1:4, or about 1:2 to about 1:3. In preferred embodiments, the microparticle has a ratio of a combination of Poly(I/C) to carrier polymer of about 1:1 to about 1:7. In more preferable embodiments, the microparticle has a ratio of a combination of Poly(I/C) to carrier polymer of about 1:1 to about 1:6. In even more preferable embodiments, the microparticle has a ratio of a combination of Poly(I/C) to carrier polymer of about 1:1 to about 1:5. In the most preferable embodiments, the microparticle has a ratio of a combination of Poly(I/C) to carrier polymer of about 1:1 to about 1:3.

In certain embodiments, the microparticle has a ratio of a combination of Poly(I/C) to carrier polymer of about 1:1, about 1:2, about 1:3, about 1:4, about 1:5, about 1:6, about 1:7, about 1:8, about 1:9, and about 1:10. In preferred embodiments, the microparticle has a ratio of a combination of Poly(I/C) to carrier polymer of about 1:1, about 1:2, about 1:3, about 1:4, about 1:5, about 1:6, or about 1:7. In more preferable embodiments, the microparticle has a ratio of a combination of Poly(I/C) to carrier polymer of about 1:1, about 1:2, about 1:3, about 1:4, about 1:5, or about 1:6. In even more preferable embodiments, the microparticle has a ratio of a combination of Poly(I/C) to carrier polymer of about 1:1, about 1:2, about 1:3, or about 1:4. In the most preferable embodiments, the microparticle has a ratio of a combination of Poly(I/C) to carrier polymer of about 1:3, about 1:2, or about 1:1.

In certain embodiments, the starch has at least about 20% amylose, such as at least about 25% amylose or at least about 30% amylose. In certain embodiments, the starch has at least about 25% amylose. In certain embodiments, the starch has about 20% amylose to about 85% amylose, such as about 20% to about 40%, about 30% to about 50%, about 40% to about 60%, about 50% to about 70%, about 60% to about 80%, about 20% to about 30%, about 25% to about 35%, about 30% to about 40%, about 35% to about 45%, about 40% to about 50%, about 45% to about 55%, about 50% to about 60%, about 55% to about 65%, about 60% to about 70%, about 65% to about 75%, about 70% to about 80%, or about 75% to about 85% amylose. In some embodiments, the starch has 0% to about 10% amylose, such as 0% to about 5%, 0% to about 4%, 0% to about 3%, 0% to about 2%, or 0% to about 1% amylose. The starch can be a high amylose starch, such as high amylose maize starch (e.g., EURYLON®). In some preferred embodiments, the starch has about 15% amylose to about 50% amylose, more preferably about 20% amylose to about 45% amylose, and most preferably about 25% amylose to about 40% amylose. In other preferred embodiments, the starch has less than about 5% amylose, such as less than 4%, less than about 3%, less than about 2%, or less than about 1% amylose. The fraction of the starch that is not amylose is preferably amylopectin, e.g., a starch that has about 25% to about 40% amylose, preferably has about 60% to about 80% amylopectin, and a starch that has 0% to about 10% amylose preferably has about 90% to 100% amylopectin.

In certain embodiments, the starch has about 15% to about 80% amylopectin, such as about 20% to about 40%, about 30% to about 50%, about 40% to about 60%, about 50% to about 70%, about 60% to about 80%, about 15% to about 25%, about 20% to about 30%, about 25% to about 35%, about 30% to about 40%, about 35% to about 45%, about 40% to about 50%, about 45% to about 55%, about 50% to about 60%, about 55% to about 65%, about 60% to about 70%, about 65% to about 75%, or about 70% to about 80% amylopectin. In some embodiments, the starch may have about 90% to 100% amylopectin, such as about 95% to about 100%, about 96% to about 100%, about 97% to about 100%, or about 98% to about 100% amylopectin. In some preferred embodiments, the starch has about 50% to about 85% amylopectin, more preferably about 55% to about 80% amylopectin, and most preferably about 60% to about 75% amylopectin. In some preferred embodiments, the starch has at least about 90% amylopectin, such as at least about 95%, about 96%, about 97%, about 98%, or at least about 99% amylopectin. The fraction of the starch that is not amylopectin is preferably amylose, e.g., a starch that has about 60% to about 70% amylopectin and preferably has about 30% to about 40% amylose, and a starch that has about 95% to 100% amylopectin and preferably has 0% to about 5% amylose.

### Composition Production and Properties

Provided herein are compositions comprising a plurality of microparticles as described herein (e.g., supra). In certain embodiments, the Dᵥ50 (=volume based 50% cumulative undersize of the particle) of the microparticles in the disclosed composition ranges from about 0.1 μm to about 200 μm, from about 0.1 μm and 100 μm, preferably from about 1 μm to about 50 μm, more preferably from about 2 μm to about 40 μm, even more preferably from about 2 μm to about 20 μm, and most preferred from about 10 μm to about 20 μm. In some embodiments, the Dᵥ50 is about 13, about 14 or about 15 μm. In other embodiments, the microparticle may have a size of about 2 μm to about 30 μm, such as about 4 μm to about 30 μm, about 5 μm to about 30 μm, or about 6 μm to about 30 μm. The microparticle may have a size of about 2 μm to about 27 μm, such as about 4 μm to about 27 μm, about 5 μm to about 27 μm, or about 6 μm to about 27 μm. The microparticle may have a size of about 2 μm to about 20 μm, such as about 4 μm to about 20 μm, about 5 μm to about 20 μm, or about 6 μm to about 20 μm. The microparticle may have a size of about 2 μm to about 10 μm, such as about 4 μm to about 10 μm, about 5 μm to about 10 μm, or about 6 μm to about 10 μm.

In preferred embodiments, a composition comprising a plurality of microparticles is stable during storage at room temperature for at least about 1 month, such as at least about 2 months, at least about 3 months, at least about 4 months, at least about 5 months, at least about 6 months, at least about 7 months, at least about 8 months, at least about 9 months, at least about 10 months, at least about 11 months, or at least about 12 months. Stability can include a lack of strand breakage and depurination/depyrimidination over time. The physical properties of the composition, such that it is powder suitable for nasal administration, is also a factor in evaluating stability.

### Administration and Dosing

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

In some embodiments, the disclosed compositions are nasally administered in the range of once per day, to once per week, to once per two weeks, to once per month. In preferred embodiments, the microparticles and compositions are administered nasally. As used herein, the term “nasally” or “nasal administration” refers to a delivery of the microparticles to the mucosa of the subject's nose such that the microparticle content is absorbed directly into the nasal tissue.

In certain embodiments, the microparticles and compositions are for pulmonary administration. As used herein, the term “pulmonary” refers to an administration through the subject's nose or mouth to deliver the microparticles to alveolar lung tissues where it is absorbed into the body. The pulmonary administration mode can be, e.g., direct inhalation of a composition, such as a powder, or inhalation of an aerosol that contains the composition. Also contemplated herein is joint nasal and pulmonary administration where a portion of the microparticles are delivered to the nasal mucosa and a portion are delivered to the alveolar lung tissues.

Certain embodiments include a nasal delivery device comprising a composition disclosed herein. The device can be a single dose nasal powder delivery device, such as those available from Aptar Pharma Germany. The unit dose device is an active delivery system, meaning that the patient does not need to inhale and performance is patient independent. Dosing is performed by actuation, which is controlled by overpressure. The dose per puff is determined by the concentration of Poly I and Poly C in the spray dried powder and the emitted weight of the powder. The powder can be administered into each nostril using a new device for each puff.

### Methods of Treatment

Provided herein are pharmaceutical compositions for use in medicine. Certain embodiments include a method of preventing upper respiratory infections, comprising administering to a subject a composition as described herein. The method can be for preventing a viral infection of the respiratory tract. The viral infection may be a human rhinovirus infection or an influenza virus infection. Other viral infections may be caused by a picornavirus (e.g., rhinovirus), coronavirus, influenza virus, human parainfluenza virus, human respiratory syncytial virus, adenovirus, enterovirus, or metapneumovirus. In certain embodiments, the composition is administered nasally to the subject. In other embodiments, the composition is administered to the subject through pulmonary inhalation.

As used herein, a therapeutic that “prevents” a condition, disorder or disease refers to a microparticle (or compositions comprising them) that, in a statistical sample, reduces the occurrence of the disorder or condition in the treated sample relative to an untreated control sample, or delays the onset or reduces the severity of one or more symptoms of the disorder or condition relative to the untreated control sample.

The microparticles and their compositions comprising them are directed to eliciting an immune response from a subject that is not necessarily infected with a virus as disclosed herein. Without wishing to be bound by theory, it is believed that administration to a subject causes TLR to recognize the Poly(I:C) and activate transcription factors like NF-κB and Interferon Regulatory Factor 3 (IRF3) to increase production of type I interferon which signal other cells to increase their antiviral defenses. When administered to the nasal mucosa, the Poly I and Poly C in the administered composition anneal and induce expression of anti-viral proteins like interferon α and β (IFNs) in the nasal epithelium. The IFNs then proceed to induce the expression of Interferon Stimulated Genes (ISGs) that further contribute to the antiviral state in the cell and stimulate adjacent cells to activate their innate response as well.

In certain embodiments, the microparticles or compositions comprising them are administered to the subject before the subject is exposed to a virus (i.e., pre-exposure prophylaxis (PrEP). The microparticles or compositions comprising them act to initiate the subject's innate viral immune response such that the virus cannot infect and replicate. In this way, the microparticles or composite ons comprising them prevent the viral infection. However, it will be understood that due to factors such as interpatient variability, dosing irregularities, or other idiosyncratic circumstances, use of a composition as disclosed herein does not necessarily result in 100% immunity in all subjects. However, because the composition, across all patients, reduces the risk of viral infection upon exposure to a virus and/or reduces the severity of an ensuing infection, the composition prevents viral infections.

In some embodiments, the microparticles or compositions comprising them are administered to the subject on a regular basis, such as once a month, once every six months, or once a year, to maintain the subject's immune response to the viruses as disclosed herein. The subject's multi-pathway immune response to the microparticles and compositions comprising them creates a durable biological defense mechanism that prevents (e.g., reduces the risk of) the subject contracting a viral infection as described herein.

In certain embodiments, a subject may be suspected of or known to being prone to suffer from viral infections as defined herein. This subject may have a susceptibility or predisposition for viral infections, including but not limited to hereditary predisposition. Such a predisposition can be determined by standard assays, using, for example, genetic markers or phenotypic indicators. Thus, the term “preventing” includes the use of a composition as disclosed herein in a subject before any clinical and/or pathological symptoms are diagnosed or determined or can be diagnosed or determined by the attending physician.

Provided herein are methods for preventing upper respiratory infections, comprising nasally administering to a subject a composition, wherein

the composition comprises a plurality of microparticles; and

the microparticles comprise polyinosinic acid and polycytidylic acid mixed with one or more carrier polymers.

Provided herein are methods for preventing upper respiratory infections, comprising nasally administering to a subject a composition, wherein

the composition comprises a plurality of microparticles; and

the microparticles comprise polyinosinic acid, polycytidylic acid, and one or more carrier polymers comprising starch, hyaluronate, alginate, carboxymethyl cellulose, microcrystalline cellulose, and/or dipalmitoylphosphatidylcholine.

In other embodiments, provided herein are uses of a disclosed composition for the manufacture of a medicament for preventing upper respiratory infections by intranasal administration of the composition. The medicament can be for preventing a viral infection of the respiratory tract. Non-limiting examples of viral infections include a human rhinovirus infection or an influenza virus infection.

In certain embodiments, provided herein is a use of a composition for the manufacture of a medicament for preventing upper respiratory infections by intranasal administration of the composition, wherein

the composition comprises a plurality of microparticles; and

the microparticles comprise polyinosinic acid and polycytidylic acid mixed with one or more carrier polymers.

In certain embodiments, provided herein is a use of a composition for the manufacture of a medicament for preventing upper respiratory infections by intranasal administration of the composition, wherein

the composition comprises a plurality of microparticles; and

the microparticles comprise polyinosinic acid, polycytidylic acid, and one or more carrier polymers comprising starch, hyaluronate, alginate, carboxymethyl cellulose, microcrystalline cellulose, and/or dipalmitoylphosphatidylcholine.

Provided herein is a nasal delivery device comprising a composition, wherein

the composition comprises a plurality of microparticles; and

the microparticles comprise polyinosinic acid and polycytidylic acid mixed with a carrier polymer and water.

Provided herein is a nasal delivery device comprising a composition, wherein

the composition comprises a plurality of microparticles; and

the microparticles comprise polyinosinic acid, polycytidylic acid, and one or more carrier polymers comprising starch, hyaluronate, alginate, carboxymethyl cellulose, microcrystalline cellulose, and/or dipalmitoylphosphatidylcholine.

Viral infections of the respiratory system can be particularly serious in patients with chronic or congenital dysfunction of the respiratory system, such as asthma, cystic fibrosis, or chronic obstructive pulmonary disease (COPD). Accordingly, in any of the disclosed methods, the subject may have chronic obstructive pulmonary disease (COPD), asthma cystic fibrosis, or another condition that results in compromised respiratory function compared to a healthy subject. Subjects with lung cancer may also receive the disclosed compositions. In certain embodiments, the subjects are smokers, with either or both of a past history of smoking or an ongoing use of cigarettes or other smoking products. These subjects are vulnerable to upper respiratory infections, so administration of a disclosed composition could potentially prevent upcoming common cold symptoms or illnesses and thus prevent exacerbations of their underlying illnesses and symptoms.

In certain embodiments, the composition administered to the subject comprises a plurality of microparticles that contain both Poly I and Poly C, either admixed within microparticles of the composition or present singly in different particles admixed in the composition, though preferably the former.

In some embodiments where disclosed compositions that contain both Poly I and Poly C are nasally administered to a subject, the Poly I and Poly C remain single-stranded until they contact the nasal mucosa. In other embodiments, a fraction of the Poly I and Poly C interact to form Poly(I:C) in the composition. In certain embodiments where formation of Poly(I:C) occurs using intranasal administration, the composition includes a buffer, such as phosphate buffered saline, and a cation, such as sodium. The ionic strength of such a composition promotes formation of double-stranded nucleic acid molecules.

### EXAMPLES

### Example 1: Comparison of Poly(I:C) vs. Poly I+Poly C Powder Preparations

The IFN-β inducing ability of a dry powder Poly(I:C) preparation was compared with that of a mixture of Poly I and Poly C.

### Mice

The generation of the IFN-β reporter mice has been previously described (Lienenklaus et. al. J. Immunol. 2009 183:3229-36). Briefly, the mice produce firefly luciferase driven by the IFN-β promoter due to a targeted mutation in the IFN-β locus. The reporter mice used in this study were heterozygous IFN-β^{+/Δβ-luc} albino (Tyr^{c2J}) C57BL/6.

### Administration of Compounds

Dry powder Poly(I:C), Poly I and Poly C was provided by Janssen.
Poly I/pea starch Ratio 1.04/12
Poly C/pea starch Ratio 0.96/12
Poly(I:C)/pea starch Ratio 1/12

A single-stranded Poly I and a single-stranded Poly C combination powder was prepared by intensive vortexing of a 1+1 mixture of Poly I and Poly C. The powders were administered to mice under injection anesthesia. The administration was done with a spatula to cover the complete nose for the time of anesthesia. This administration resulted in a detectable up-regulation of IFN-β (luciferase) in known experiment procedures.

To further quantify the assays, administration in two follow up experiments used self-made “tip-devices” to administer 2 mm of powder into both nostrils (in 3) or 2×2 mm into the left nostril (in 4) or 2×2 mm into the left nostril and an additional 2mm onto the nose (in 4).

### Imaging of Mice

For in vivo imaging, mice were i.v. injected with D-Luciferin, firefly, potassium salt (30 mg/ml in PBS; 100 μl/20 g mouse). Mice were anesthetized with Isoflurane and photon emission was monitored using an IVIS200 system (CaliperLS) ˜5 min after injection of luciferin.

Data correction for the time span between luciferin administration and imaging was performed as follows: Corrected flux=total flux+Δt[min]*(0.0459*total flux). Imaging was performed 24 h after powder administration.

### Results

Eleven mice were dosed with 2 mm of the following powders using a spatula according to Table 1.

TABLE 1

| Number | Powder |
|---|---|
| of mice | administered |
| 2 | Poly I |
| 2 | Poly C |
| 3 | Poly (I:C) |
| 4 | Poly I + Poly C |

FIG. 1 indicates that Poly I+Poly C induced comparable amounts of IFN-β to Poly(I:C). However, the assay signals were close to baseline.

Seventeen mice were dosed with the following powders using a 2 mm tip-device in both nostrils of the mice according to Table 2. I+C mix was freshly prepared: 0.0261 g I (1.04/12)+0.0271 g C (0.96/12). Table 2 indicates the amount of powder that was administered to each nostril of a given mouse. For example, mouse 1 in the first treatment received 1.5 mm in the left nostril and 1.5 mm in the right nostril of Poly(I:C) on Day 1. Mouse 1 then received 1.5 mm in the left nostril and 2 mm in the right nostril of Poly(I:C) on Day 2.

TABLE 2

| | 1st | 2nd |
|---|---|---|
| Mouse | treatment | treatment |
| 1 | I:C 1.5 + 1.5 | 1.5 + 2 |
| 2 | I:C 2 + 1 | 1.5 + 1.5 |
| 3 | I:C 2 + 1.5 | 2 + 2 |
| 4 | I:C 1.5 + 2 | 2 + 2 |
| 5 | I:C 2 + 2 | 2 + 2 |
| 6 | I:C 2 + 2 | 2 + 2 |
| 7 | I + C 2 + 2 | 2 + 2 |
| 8 | I + C 2 + 1.5 | 2 + 2 |
| 9 | I + C 2 + 1.5 | 2 + 2 |
| 10 | I + C 2 + 2 | 2 + 2 |
| 11 | I + C 2 + 1.5 | 2 + 2 |
| 12 | I + C 3 + 1 | 2 + 2 |
| 13 | I 2 + 1 | 1 + 2 |
| 14 | I 1.5 + 1.5 | 2 + 1 |
| 15 | C 1.5 + 1.5 | 2 + 2 |
| 16 | C 1.5 + 2 | 2 + 2 |
| 17 | C 2 + 1 | 1.5 + 2 |

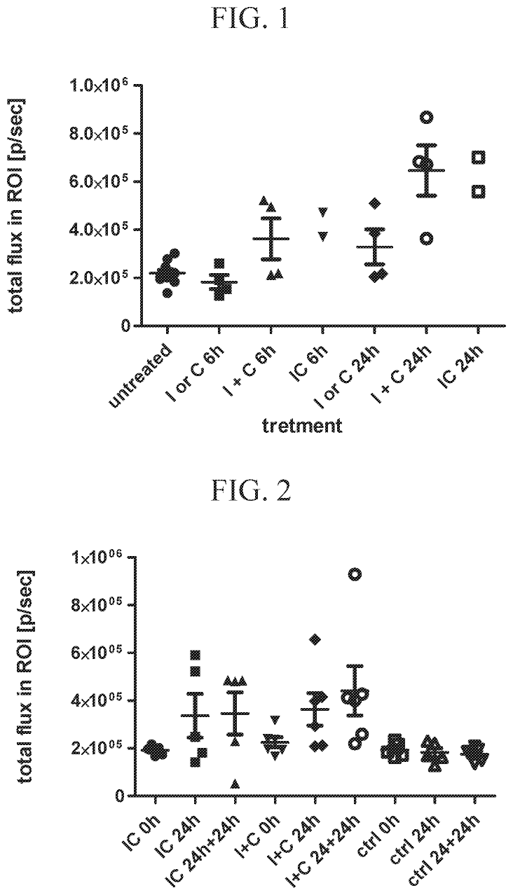

FIG. 2 indicates equivalent IFN-β signal induction for all powders assayed (one outlier signal excluded). Re-administration of powder on Day 2 did not result in significantly elevated signals. No elevated signal occurred in the control group (differently treated mice were kept separated).

Twenty-two mice were dosed with the following powders using a 2 mm tip-device in the left nostril according to Table 3. Several mice received an additional dose directly on the nose-tip.

TABLE 3

| | | Left | Additional |
|---|---|---|---|
| Mouse | Treatment | nostril | onto nose |
| 1 | I:C | 2 + 2 | 2 |
| 2 | I:C | 2 + 2 | 2 |
| 3 | C | 1.5 + 2 | 2 |
| 4 | I + C | 2 + 2 | 2 |
| 5 | I + C | 2 + 2 | 2 |
| 6 | C | 1 + 1 | 2 |
| 7 | I + C | 2 + 2 | 2 |
| 8 | I:C | 2 + 2 | 2 |
| 9 | I + C | 2 + 2 | 2 |
| 10 | I:C | 2 + 2 | 2 |
| 11 | I + C | 2 + 2 | 2 |
| 12 | I:C | 2.5 + 2 | — |
| 13 | I + C | 2 + 1 + 1 | — |
| 14 | C | 2 + 2 | 2 |
| 15 | I:C | 1 + 2 | — |
| 16 | I + C | 2 + 2 | — |
| 17 | C | 2 + 2 | 2 |
| 18 | I:C | 2 + 2 | — |
| 19 | I:C | 2 + 2 | — |
| 20 | I + C | 1 + 1 + 1 | — |
| 21 | I + C | 2 + 2 | — |
| 22 | C | 2 + 2 | 2 |

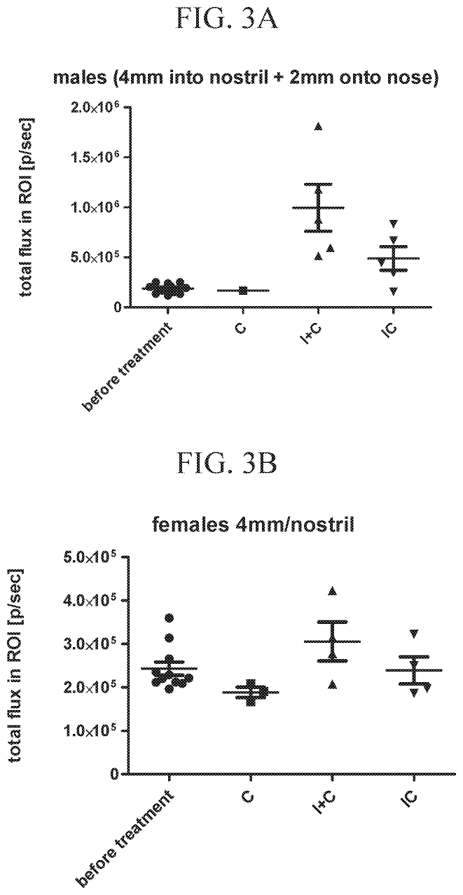

FIG. 3A and FIG. 3B depict the IFN-β signal induction in mice who were nasally administered a composition of Poly I, Poly C, Poly(I:C) or mixed Poly I+Poly C initially in the left nostril only, with male mice in FIG. 3A receiving an additional dose on their nose-tip. The figures illustrate that one nostril only administration does not result in detectable IFN-β induction.

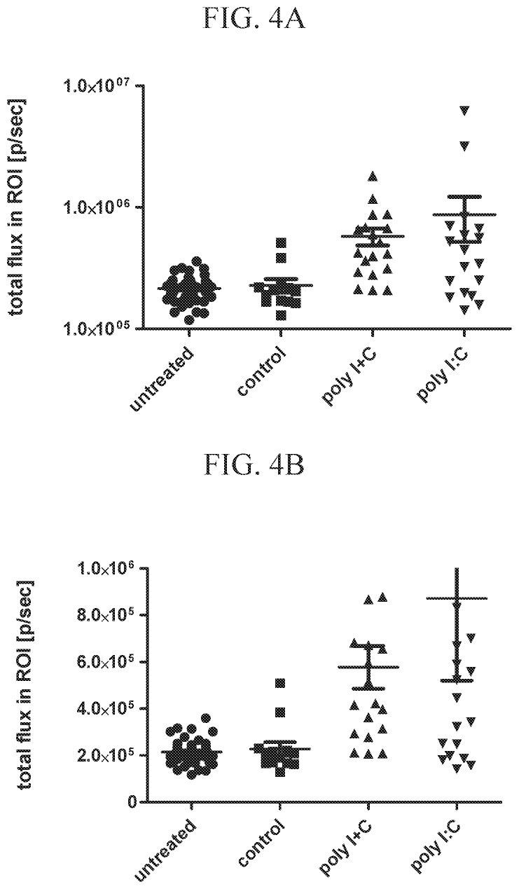

Taken as a whole, the three cohorts of mouse data compiled in FIG. 4A (logio scale of IFN-β induction signal) and FIG. 4B (linear view of IFN β induction signal) indicate that dry powder Poly(I:C) and a mix of dry powder Poly I and dry powder Poly C induce IFN-β equally well.

### Example 2: Effect of Molecular Weight on Immune Response

The IFN-β inducing ability of standard-sized Poly(I:C) (“standard Poly(I:C)”) (Sigma-Aldrich, >300 kDa) was compared to that of a lower molecular weight (“LMW Poly(I:C)”) sized Poly(I:C) (Invivogen, Catalog #t1r1-picw, 66-305 kDa

### Mice

The generation of the IFN-β reporter mice has been previously described (Lienenklaus et. al. 2009). Briefly the mice produce firefly luciferase driven by the IFN-β promoter due to a targeted mutation in the IFN-β locus. The reporter mice used in this study were heterozygous IFN-β^{+/Δβ-luc} albino (Tyr^{c2}J) C57BL/6. For analyzing the induction pathways these mice were crossed with several knockout mice. (IRF3^{-/-} IRF7^{-/-} and IFN-b^{-/-} in this study)

### Administration of Compounds

Standard Poly(I:C) was provided as a standard and dissolved in PBS at different concentrations. LMW Poly(I:C) was also dissolved in PBS at different concentrations. The preparation of diluted solutions is given in Table 4. The “mice” column indicates how many mice were administered that solution. The standard Poly(I:C) was heated to 65° C. to facilitate dissolution of the dsRNA.

TABLE 4

| mice | 65° C. heated samples | | | | mice | LMW (Invivogen) | | |
|---|---|---|---|---|---|---|---|---|
| 10 | 30 μg | 300 μl | Stock (2 mg/ml) | | 8 | 300 μg | 200 μl | Stock (20 mg/ml) |
| 2 | 20 μg | 50 μl + 25 μl | | | 6 | 30 μg | 20 μl + 180 μl | |
| 4 | 10 μg | 60 μl + 120 μl | | | 4 | 3 μg | 20 μl + 180 μl | |
| 2 | 6 μg | | 30 μl + 20 μl | | | | | |
| 4 | 3.3 μg | | 50 μl + 100 μl | | | | | |
| 4 | 1.1 μg | | | 50 μl + 100 μl | | | | |

15 μl of the dilution were administered nasally to the mice within ˜2 min (4 droplets, 3.8 μl each) under isoflurane anesthesia.

### Imaging of Mice

For in vivo imaging mice were i.v. injected with D-Luciferin, firefly, potassium salt (30 mg/ml in PBS; 100 μl/20 g mouse). Mice were anesthetized with Isoflurane and photon emission was monitored using an IVIS200 system (CaliperLS) ↦5 min after injection of luciferin. In this experiment images were taken before and 3, 6, 24, 100 h after compound administration.

Data correction for the time span between luciferin administration and imaging was performed as follows: Corrected flux=total flux+Δt[min]*(0.0481*total flux−7580.9).

### Results

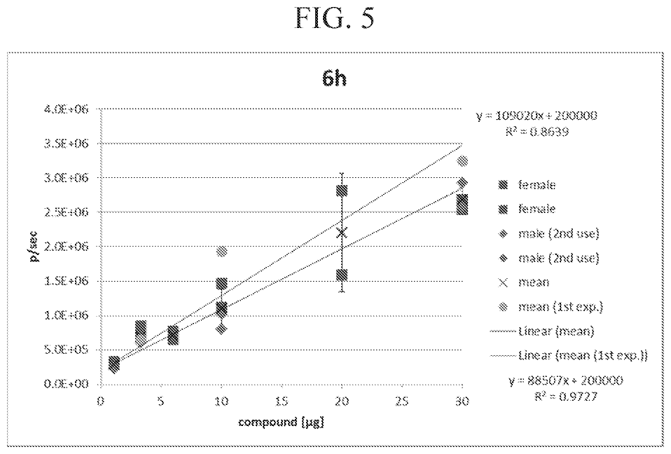

A dose response curve was generated from the 6 h data as shown in FIG. 5. Female mice (square) and male mice (diamond) showed a similar response to standard Poly(I:C) administration. The circle symbols indicate the means of the previous dose response with the standard Poly(I:C). Comparing the slopes gives an inter assay variation of these first two dose response experiments of ˜20%.

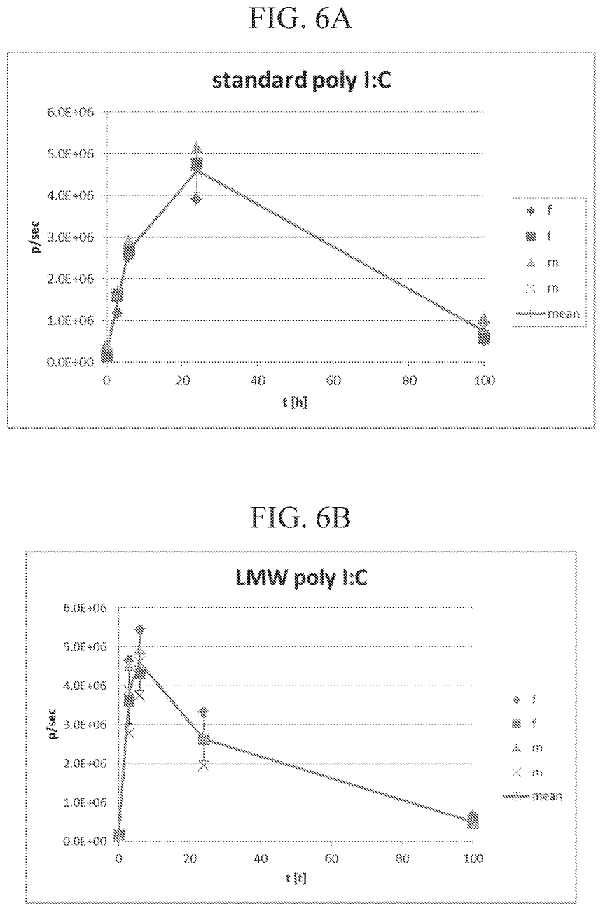

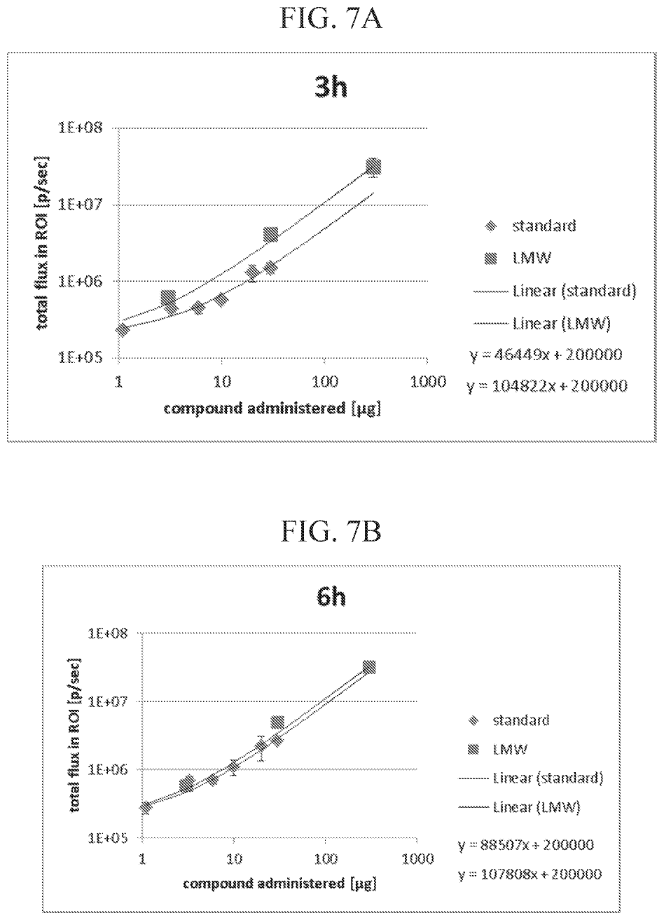

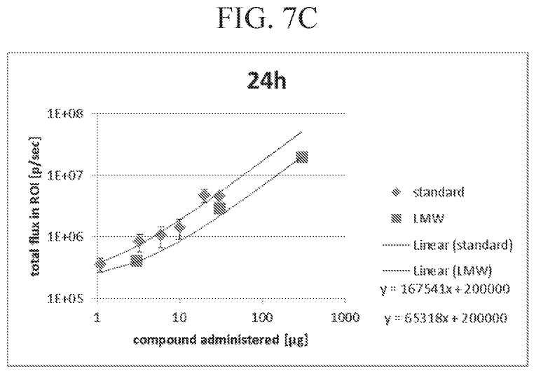

Comparing the kinetics of IFN-β induction after administration of standard Poly(I:C) and LMW Poly(I:C) reveals a slower up regulation and a more sustained signal in the standard Poly(I:C) induced mice. FIG. 6A showed the kinetics of IFN-β induction after administration of standard Poly(I:C), and FIG. 6B depicts the kinetics of IFN-β induction after administration of LMW Poly(I:C).

To include a marker for the type of IFN-β response in subsequent studies, a 6 h/24 h ratio was evaluated. A ratio <1 indicated a standard Poly(I:C)like action while a ratio >1 indicated an LMW Poly(I:C) type of action.

The slopes of the dose response curves for standard Poly(I:C) and LMW Poly(I:C) were calculated from the data collected at 3 h (FIG. 7A), 6 h (FIG. 7B) and 24 h (FIG. 7C). The graphs show the means, standard deviation, and linear regression (fixed background at 2e5 p/sec) on logarithmic scale. At 3 h the LMW Poly(I:C) slope is higher, at 6 h the two slopes are comparable (LMW Poly(I:C) slightly higher), at 24 h the standard Poly(I:C) dose response curve has a higher slope.

The kinetics of the induction in IFN-β reporter mice carrying knockout mutations of molecules involved in the positive feedback process were studied to further understand the differences observed when comparing standard Poly(I:C) and LMW Poly(I:C). Positive feedback is a type of regulatory process in the body. Here, signal transduction immediately after ligand recognition of Poly(I:C) in TLR3 depends on constitutively expressed IRF3. A first wave of immediate early Type I IFNs are released (IFN-β and IFNα4). Via IFNAR signaling based on the IFN increase, IRF7 is upregulated. Together with IRF3, IRF7 induces a second wave of IFN production dependent upon the availability of the ligand. The duration of the availability of the signal can be a difference when comparing formulations having LMW Poly(I:C) and standard Poly(I:C).

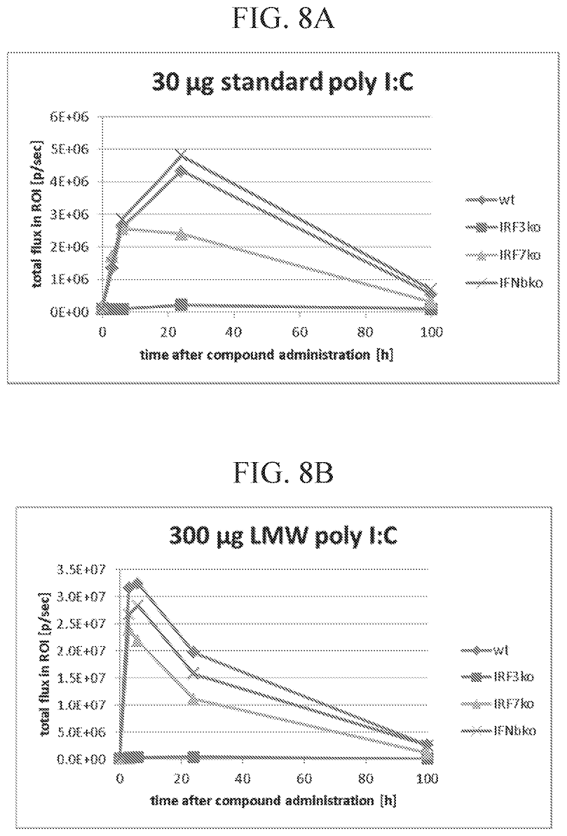

FIG. 8A shows the IFN-β reporter induction at the nose in wt IFN-β reporter mice as well as in reporter mice lacking IRF3, IRF7 or IFN-β. These mice were dosed with 30 μg standard Poly(I:C). FIG. 8B shows this data for mice dosed with 300 μg of LMW Poly(I:C). In a study of dosing 30 μg LMW Poly(I:C), wt, IRF3 and IFN-b ko mice showed the same curve shapes as 300 μg treated mice.

At all time points after administration, the signal significantly depends on IRF3 levels. At 24 h after administration, IRF7ko mice show only about half of the signal of wt mice. Without wishing to be bound by any theory, the data suggests a role for positive feedback in the response to standard Poly(I:C). Interestingly, this feedback does not depend on IFN-β. Typically IFN-β is the major early Type I IFN. IFNα4 (or other mechanisms) might compensate in this context.

The induction of the IFN-β reporter by LMW Poly(I:C) depends on IRF3 levels as is seen with standard Poly(I:C). In contrast to standard Poly(I:C), LMW Poly(I:C) induced IFN-β shows no dependence on IRF7 at 24 h after administration.

### Example 3. Methods of Preparing and Characterizing Microparticles

### Spray Drying of Poly(LC) with Pea Starch

The spray dry process was performed on a Buchi B290 Mini spray dryer (Buchi, Flawil, Switzerland). Nuclease free water added to a glass beaker and the pea starch is added while mixing using a magnetic stirrer until the starch is completely dispersed. Poly(I:C) was dissolved in nuclease free water and stirred on a magnetic stirrer until the Poly(I:C) is completely dissolved. The dissolved Poly(I:C) is added to the dispersed pea starch and stirred at room temperature overnight. A total solids concentration of 4.7% (w/w) and a ratio of Poly(I:C)/pea starch 1/3 (w/w) was applied.

The solutions were fed to a two-fluid nozzle (diameter: 0.7 mm) at the top of the spray dryer by means of a peristaltic pump. The spray dryer operated in co-current nitrogen flow mode. The spray dried particles were collected in a reservoir attached to a cyclone. After collection of the particles, the glass cylinder and cyclone was cooled to room temperature. The collected powder was transferred to amber glass bottle and this bottle is placed in an aluminum vapor lock bag. The vials were stored at room temperature.

### Scanning Electron Microscopy

The samples were sputtered with gold particles with diameter +/−30-50 nm. Images were generated using a FEI scanning electron microscope-type Quanta 200F with Everhart Thornley detector.

### Water Content—Karl Fischer Titration

Water content of the concepts was determined by means of a direct volumetric Karl Fisher titration. A KF TITRATOR V30 is used (Mettler Toledo, US). The powder (50-100 mg) was transferred to the titration vessel containing Hydranal® Methanol Dry (Sigma Aldrich) and stirred for 300 seconds. Titration was performed with Hydranal® Composite 2 (Sigma Aldrich) at a concentration of 2 mg/ml using a 5 ml burette. For termination, a stop drift of 15 μg/min was applied. Samples were analyzed in triplicate.

### Determination of Particle Size

There exists a tendency to evaluate particle size distribution data merely on the basis of the volume distribution of the products of interest. Thereby, one often limits the valuation to a comparison of the Dᵥ10, Dᵥ50 and Dᵥ90 cumulative undersizes. However, comparing dᵥx cumulative undersizes may not always be straight-forward due to the fact that different techniques and instruments readily lead to different results. In addition, one can get more information out of a particle size (or shape) distribution data by looking from a different perspective to the data (i.e., using other parameters).

For the determination of the particle size distribution the laser diffraction test method was used. The analysis was performed on a Malvern Mastersizer 2000 laser diffractometer equipped with a Hydro2000S wet dispersion module (or an equivalent system). The instrument is used in the blue light ON detection mode at a size range of 20 nm to 2 mm. The measured particle size distribution by volume in the current invention for Dᵥ10 is 4 μm, for Dᵥ50 it is 14 μm while for Dᵥ90 it is 27 μm.

### Example 4. In Vivo Testing of Formulations in the Influenza Mouse Model

All animal studies were approved by the ethical committee and performed according to national and international guidelines. 8-12 week old female Swiss mice (Janvier) were used. All intranasal treatments were performed under isoflurane anesthesia. To administer an amount of liquid, a droplet was applied directly on top of the nostril and, by closing the mouth, the droplet was allowed to enter via the nostril into the nasal cavity. Spray dried Poly(I:C)-pea starch powders were freshly prepared just prior to each experiment and administrated with a powder tip device. Unformulated Poly(I:C) was administrated in phosphate buffered saline (PBS) at a concentration of 1 mg/ml.

### Testing the IFN-β Inducing Capacity of PolyIC/Lycoat RS780 (=Pea Starch) in Different Ratios (1/3, 1/5, 1/12)

The luciferase reporter for interferon-beta (IFN-β) gene activation can provide insight in the activation of IFN-β after stimulation with different Poly(I:C) formulations. Poly(I:C) is a synthetic analog which mimics dsRNA viruses by stimulation of the innate immune system through Pattern Recognition Receptors (PRR). When Poly(I:C) binds to its PRR (TLR3), RIG-1 and/or MDA 5, a signaling cascade is started and results in activation of type I interferons, of which IFN-β is a representative. The heterozygous IFN-β+/Δβ-luc albino (Tyrc2J) C57BL/6 mice produce firefly luciferase driven by the IFN-β promoter due to a targeted mutation in the IFN-β locus. For optical imaging luciferin is administered systemically and photon emission is monitored using an IVIS200 system (CaliperLS).

The IFN-β inducing capacity of a compound in a dry powder formulation was tested in different ratios Poly(I:C)/Lycoat RS780 (=pea starch) 1/3, 1/5, and 1/12. A pea starch-only formulation was included as a negative control. The compounds were administered nasally to IFN-β reporter mice and in vivo imaging was performed before and 24 h after administration.

### Mice

The generation of the IFN-β reporter mice has been previously described (Lienenklaus et. al. 2009). Briefly, the mice produce firefly luciferase driven by the IFN-β promoter due to a targeted mutation in the IFN-β locus. The mice used in this study were heterozygous IFN-β+/Δβ-luc albino (Tyrc2J) C57BL/6. Males and females between the age of 12 and 14 weeks were used. The animals are housed in IVC racks and they are provided with food and water ad libitum.

### Administration of Compounds

Thirty one 8-12 week old male and female heterozygous IFN-β+/Δβ-luc albino (Tyrc2J) C57BL/6 were used. All intranasal administrations were performed under injection anesthesia (ketamine/xylazine). The dry powder was administered using a self-made tip device. The tip-devices were used to administer 2 mm of powder into the left nostril and an additional 2 mm of powder onto the nose. After the administration of the powder, the mice were placed under a red lamp to wake up.

### Imaging of Mice

For in vivo imaging mice were i.v. injected with D-Luciferin, firefly, potassium salt (30 mg/ml in PBS; 100 μl/20 g mouse). Mice were anesthetized with isoflurane, and photon emission was monitored using an IVIS200 system (CaliperLS) ˜5 min after injection of luciferin. Imaging was performed 4 hours before (background signal) and 24 hours after compound administration. Data correction for the time span between luciferin administration and imaging was done: Corrected flux=total flux+Δt[min]*(0.0459*total flux).

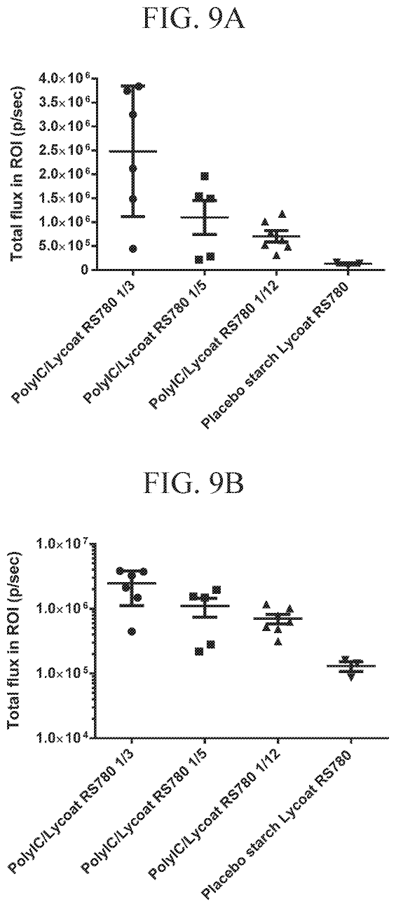

Statistical analysis of the actual data showed a significant difference between the groups Poly(I:C)/Lycoat RS780 (=pea starch) 1/3 versus 1/12 and also between the Poly(I:C)/Lycoat RS780 1/3 versus the Placebo starch Lycoat RS780 group (FIG. 9A and FIG. 9B). The statistical analysis of the log transformed data showed a significant difference between the groups Poly(I:C)/Lycoat RS780 1/3 versus Placebo starch Lycoat RS780 group, the 1/5 group versus the placebo group and the 1/12 group versus the placebo group (FIG. 10A and FIG. 10B). In conclusion, there is a good correlation between the activity of the formulation (IFN response) and the ratio of Poly(I:C)/Lycoat RS780 in the formulation. The IFN response increases when a higher ratio of Poly(I:C)/Lycoat RS780 starch is used.

### Example 5: Efficacy Study in Human Subjects

This trial studied the efficacy of PrEP-001, a dry powder microparticulate composition, in which the microparticles are a mixture of 400 μg polyinosinic acid, 400 μg polycytidylic acid, and 10 mg pregelatinized waxy maize starch with a certain amount of residual moisture.

In this trial, 44 healthy subjects who did not exhibit antibodies to a human rhinovirus (HRV) strain (HRV-16) were selected and randomized so that one group received two doses of PrEP-001 48 and 24 hours before exposure to HRV-16 and the other group received a placebo (10 mg spray-dried pregelatinized waxy maize starch) at those time points. The doses were administered nasally to all subjects.

As shown in FIG. 11A, treatment with PrEP-001 reduced the duration and severity of symptoms compared with placebo. The median symptom duration was reduced from 6.0 to 1.7 days. The percentage of PrEP-001-treated subjects classified as ill was lower than in the placebo-treated subjects as shown in FIG. 11B. Clinical illness was defined as positive for virus and a total symptom score >2 for two consecutive days. The modified Jackson questionnaire was used to evaluate the symptoms experienced by patients. The Modified Jackson score was calculated by summing 10 symptom scores (sneezing, headache, malaise, chilliness, nasal discharge, nasal obstruction, sore throat and cough, muscle aches and fever), rated as 0=absent, 1=mild, 2=moderate, and 3=severe. There were three questionnaires a day for 7 days plus one prior to discharge on day 8.

In addition, subjects that were administered the placebo prior to exposure to the HRV strain were over three times more likely to be classified as clinically ill with a cold than subjects who received PrEP-001. As shown in FIG. 12, the percentage of PrEP-001-treated subjects classified as clinically ill after exposure was 23% (5 of 22 subjects), while the percentage of the placebo-treated subjects classified as clinically ill after exposure was 73% (16 of 22 subjects). Thus, administration of PrEP-001 reduced the number of subjects that were both positive for viral infection and developed symptoms of a cold in this study.

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A microparticle comprising polyinosinic acid and polycytidylic acid mixed with one or more carrier polymers.

2. The microparticle of any preceding claim, wherein the one or more carrier polymers comprise one or more of starch, hyaluronate, alginate, carboxymethyl cellulose, microcrystalline cellulose, or dipalmitoylphosphatidylcholine.

3. The microparticle of any preceding claim, wherein the one or more carrier polymers comprise alginate, e.g., sodium alginate.

4. The microparticle of any preceding claim, wherein the one or more carrier polymers comprise dipalmitoylphosphatidylcholine.

5. The microparticle of any preceding claims, wherein the one or more carrier polymers comprise starch.

6. The microparticle of claim 5, wherein the starch comprises one or more starches selected from maize starch, cornstarch, wheat starch, potato starch, pea starch, banana starch, rice starch, barley starch, rye starch, millet starch, oat starch, yam starch, sweet potato starch, cassava starch, tapioca starch, sago starch, arrowroot starch, fava bean starch, lentil starch, mung bean starch, and chickpea starch.

7. The microparticle of claim 5, wherein the starch comprises a maize starch, pea starch, potato starch, or cassava starch.

8. The microparticle of any one of claims 5-7, wherein the starch comprises a pregelatinized starch or a partially pregelatinized starch.

9. The microparticle of claim 8, wherein the pregelatinized starch or a partially pregelatinized starch is partially pregelatinized maize starch, pregelatinized pea starch, or pregelatinized potato starch.

10. The microparticle of any one of claims 5-8, wherein the starch comprises pea starch.

11. The microparticle of claim 10, wherein the pea starch is hydroxypropylated pregelatinized pea starch.

12. The microparticle of any one of claims 5-7, wherein the starch comprises one or more starches selected from dextrin, acid-treated starch, alkaline-treated starch, bleached starch, oxidized starch, enzyme-treated starch, monostarch phosphate, distarch phosphate, phosphated distarch phosphate, acetylated distarch phosphate, starch acetate, acetylated distarch adipate, hydroxypropyl starch, hydroxypropyl distarch phosphate, hydroxypropyl distarch glycerol, starch sodium octenyl succinate, starch aluminum octenyl succinate, acetylated oxidized starch, cationic starch, hydroxyethyl starch, or carboxymethylated starch.

13. The microparticle of any one of claims 5-12, wherein the starch has about 20% amylose to about 85% amylose.

14. The microparticle of claim 13, wherein the starch has about 25% amylose to about 40% amylose.

15. The microparticle of any one of claims 5-12, wherein the starch has about 15% to about 80% amylopectin.

16. The microparticle of claim 15, wherein the starch has about 60% to about 80% amylopectin.

17. The microparticle of any preceding claim, wherein the ratio of the combination of polyinosinic acid and polycytidylic acid to carrier polymer ranges from 1:1 to 1:10.

18. The microparticle of any one of claims 1-16, wherein the ratio of the combination of polyinosinic acid and polycytidylic acid to carrier polymer ranges from 100: lto 1:1.

19. The microparticle of any one of claims 1-17, wherein the ratio of the combination of polyinosinic acid and polycytidylic acid to carrier polymer ranges from 1:1 and 1:3.

20. The microparticle of any preceding claim, wherein the ratio of the combination of polyinosinic acid and polycytidylic acid to carrier polymer is 1:1.

21. The microparticle of any preceding claim, wherein the microparticle comprises two or more carrier polymers.

22. The microparticle of any preceding claim, wherein the microparticle is produced by a spray-dry particle formation process.

23. The microparticle of any preceding claim, wherein the polyinosinic acid and polycytidylic acid are each present as a sodium salt.

24. The microparticle of any preceding claim, further comprising water.

25. The microparticle of any preceding claim, wherein the microparticle consists essentially of polyinosinic acid, polycytidylic acid, the one or more carrier polymers, and water.

26. The microparticle of any preceding claim, wherein the average chain length of each of the polyinosinic acid and polycytidylic acid is approximately 500 bases to 2,000 bases.

27. A composition comprising a plurality of microparticles comprising polyinosinic acid and one or more carrier polymers, and a plurality of microparticles comprising polycytidylic acid and one or more carrier polymers.

28. A composition comprising a plurality of microparticles according to any preceding claim.

29. The composition of claim 28, wherein the Dv50 of the microparticles ranges from 0.1 μm to 200 μm.

30. The composition of claim 29, wherein the Dv50 of the microparticles ranges from 0.1 μm and 100 μm.

31. The composition of claim 30, wherein the Dv50 of the microparticles ranges from 1 μm to 50 μm.

32. The composition of claim 31, wherein the Dv50 of the microparticles ranges from 2 μm to 30 μm.

33. The composition of claim 32, wherein the Dv50 of the microparticles ranges from 2 μm to 20 μm.

34. The composition of claim 33, wherein the Dv50 of the microparticles ranges from 10 μm to 20 μm.

35. The composition of any one of claims 28-34, wherein the composition is a dry powder.

36. The composition of any one of claims 28-35, wherein the composition is suitable for intranasal administration.

37. The composition of any one of claims 28-36, wherein the composition is suitable for pulmonary administration.

38. A composition of any one of claims 28-37, for use in medicine.

39. A method of preventing upper respiratory infections, comprising administering to a subject the composition of any one of claims 28-38.

40. The method of claim 39, wherein the method is for preventing a viral infection of the respiratory tract.

41. The method of claim 40, wherein the viral infection is a human rhinovirus infection or an influenza virus infection.

42. The method of claim 40, wherein the viral infection is caused by a picornavirus (e.g., rhinovirus), coronavirus, influenza virus, human parainfluenza virus, human respiratory syncytial virus, adenovirus, enterovirus, or metapneumovirus.

43. The method of any one of claims 39-42, wherein the subject has a disease selected from chronic obstructive pulmonary disease, asthma, cystic fibrosis, and lung cancer.

44. The method of any one of claims 39-43, wherein the subject has a past history of smoking or is a current smoker.

45. The method of any one of claims 39-44, wherein administering the composition comprises intranasal administration.

46. The method of any one of claims 39-45, wherein administering the composition comprises pulmonary administration.

47. Use of a composition according to any one of claims 28-38, for the manufacture of a medicament for preventing upper respiratory infections by intranasal administration of the composition.

48. Use of a composition according to claim 47, for the manufacture of a medicament for preventing a viral infection of the respiratory tract.

49. Use of a composition according to claim 48, wherein the viral infection is a human rhinovirus infection or an influenza virus infection.

50. Use of a composition according to claim 48, wherein the viral infection is caused by a picornavirus, rhinovirus, coronavirus, influenza virus, human parainfluenza virus, human respiratory syncytial virus, adenovirus, enterovirus, or metapneumovirus.

51. A nasal delivery device, comprising the composition of any one of claims 28-38.
